(19) United States
(12) Patent Application Publication
(10) Pub. No.: US 2021/0007673 A1
(43) Pub. Date: Jan. 14, 2021

(54) METHOD AND SYSTEM FOR DETECTING CONTACT BETWEEN AN OPTICAL PROBE AND TISSUE AND AUTOMATING TISSUE MEASUREMENT

(71) Applicants: Northwestern University, Evanston, IL (US); American BioOptics, LLC, Chicago, IL (US); NorthShore University HealthSystem, Eanston, IL (US)

(72) Inventors: Vadim Backman, Chicago, IL (US); David S. Mueller, Chicago, IL (US); Andrew J. Cittadine, Chicago, IL (US); Sarah Ruderman, Englewood, CO (US); Hemant Roy, Chestnut Hill, MA (US); Bradley Gould, Evanston, IL (US)

(21) Appl. No.: 16/319,669

(22) PCT Filed: Jun. 21, 2017
(86) PCT No.: PCT/US2017/038529

§ 371 Date: Jan. 22, 2019

Related U.S. Application Data
(60) Provisional application No. 62/365,681, filed on Jul. 22, 2016.

(51) Int. Cl. A61B 5/00; A61B 5/1459

(52) U.S. Cl. CPC A61B 5/6885; A61B 5/14546; A61B 5/1459

(57) ABSTRACT

Methods and apparatus initiate a procedure performed by a probe on tissue. A signal is obtained from the probe, the signal varying with changes in a proximity of the probe to the tissue. The obtained signal is analyzed by a controller to determine whether the probe is within a predetermined distance of the tissue. The controller initiates the procedure when it is determined that the probe is within the predetermined distance of the tissue. The obtaining and analyzing may be repeated until the probe is determined to be within the predetermined distance of the tissue. The analyzing may include comparing the obtained signal to a predetermined threshold.

This invention was made with government support under grant no. R01 CA109861 and grant no. R01 CA128641, both awarded by the National Institutes of health. The government has certain rights in the invention.

### BACKGROUND

Currently, there are devices available for minimally invasive in vivo diagnostic or therapeutic procedures. Many of these devices include systems with fiber-optic probes to transmit light to and from the tissue. These probes must be placed in contact with the target tissue in order to measure optical properties or to perform a therapeutic application. This usually involves applying a gentle pressure to ensure and maintain good contact and eliminate gaps between the probe and a tissue interface. Additionally, there is often a control system involved which requires a user interface for a user to initiate the activity (measurements or therapeutic application) once sufficient probe-to-tissue contact has been achieved. This interface and required user action can result in time delays between initial probe contact with the tissue of interest and initiation of the desired activity. Such factors (time delay and pressure application) can impact the physiological parameters of the tissue and therefore the optical properties measured from the tissue.

It has been demonstrated that pressure application and contact time between a probe and tissue can have a significant impact on the physiological properties of the tissue. When a firm pressure is applied by the probe to the tissue, there is an increase in the total hemoglobin content at a superficial depth of approximately 100 μM below the tissue surface and a decrease in the total hemoglobin content at a deeper depth of approximately 200 μm. There also is a significant decrease in the packaging length scale (PLS), which is proportional to blood vessel diameter, at the deeper depth of penetration when firm pressure is applied. These effects are illustrated in FIG. 9, which illustrates the pressure normalized effect of gentle and firm pressure on the following physiological parameters: hemoglobin concentration, oxygenation percentage, and the PLS %.

The effect of contact time may be amplified when a firm pressure, for example 0.15-0.2 N/mm², is applied as compared to when applying a gentle pressure. As illustrated in FIG. 10, all parameters at both depths of penetration below the tissue surface, 100 μm and 200 μm, remain within 10% of the first measurement when gentle pressure is applied. FIG. 10 shows the time normalized effect of gentle and firm pressure on the following physiological parameters over time: hemoglobin concentration, oxygenation percentage, and the PLS %. For both depths at firm pressure, the total hemoglobin content decreases over time. Oxygenation follows the same trend, although it decreases more rapidly. PLS remains relatively constant to gentle pressure at the superficial depth, and only slightly decreases at the deeper depth of penetration.

### SUMMARY

The present invention relates generally to the detection of contact between a measurement apparatus and tissue, and in particular to in vivo methods of detecting contact between a probe and tissue or detecting close proximity of a probe to tissue, and to systems to implement the methods.

In one embodiment, the present invention includes an apparatus for initiating a procedure on tissue, the apparatus comprising a probe including a transmitter that transmits a signal and a receiver that receives a reflected/diffused signal, the reflected/diffused signal varying from the transmitted signal with changes in a proximity of the probe to the tissue, and a controller that analyzes the reflected/diffused signal to determine whether the probe is within a predetermined distance from the tissue. In such an embodiment, the controller initiates the procedure when it is determined that the probe is within the predetermined distance of the tissue. The controller may include a processor and a memory. The apparatus may further comprise an output unit that, when the controller determines that the procedure has been completed, the controller (i) outputs via the output unit an instruction to an operator of the apparatus to move the probe away from the tissue, (ii) analyzes the signal as the probe is being moved away from the tissue to determine when the probe has come out of contact with the tissue, and (iii) outputs via the output unit an indication to the operator that the probe has come out of contact with the tissue.

In another embodiment, the present invention includes a method of initiating a procedure performed by a probe on tissue, the method comprising obtaining a signal from the probe, the signal varying with changes in a proximity of the probe to the tissue, analyzing the obtained signal by a controller to determine whether the probe is within a predetermined distance of the tissue, and the controller initiating the procedure when it is determined that the probe is within the predetermined distance of the tissue. In such an embodiment, the obtaining and analyzing may be repeated until the probe is determined to be within the predetermined distance of the tissue. The analyzing may include comparing the obtained signal to a predetermined threshold.

In one embodiment, the probe is determined to be in close proximity of the tissue when a value of the obtained signal is greater than the predetermined threshold. The probe may include a receiver that measures a physical property to output the obtained signal. The receiver may be used to perform the procedure. The procedure may be a diagnostic procedure.

In some embodiments, the receiver is an optical receiver and the physical property is a light intensity. In another embodiment, the receiver is an electrical receiver and the physical property is a resistivity. In yet another embodiment, the receiver is a mechanical receiver and the physical property is a pressure.

In some embodiments, the probe may transmit light onto the tissue and receive reflected/diffused light from the tissue to obtain the signal, the signal may represent an intensity of the reflected light, and the controller compares the obtained signal to a predetermined threshold intensity value. In such embodiments, the probe may receive the reflected/diffused light at multiple wavelengths, and the signal may represent an average of intensity values that are obtained from the probe according to the reflected/diffused light at the multiple wavelengths.

In other embodiments, the probe may transmit a first polarized light and a second polarized light different from the first polarized light onto the tissue and receives a first reflected/diffused light and a second reflected/diffused light different from the first reflected/diffused light from the tissue. The obtained signal may represent a ratio between the first reflected/diffused light and the second reflected light, and the controller may compare the obtained signal to a predefined threshold ratio value.

In other embodiments, after the controller determines that the procedure has been completed, the controller indicates that the procedure has been completed to cause an operator of the probe to move the probe away from the tissue, the controller analyzes the signal as the probe is being moved away from the tissue to determine when the probe has come out of contact with the tissue, and the controller indicates to the operator that the probe has come out of contact with the tissue. In such embodiments, the controller compares the obtained signal to a first predetermined threshold, and the controller compares the obtained signal to a second predetermined threshold that is different from the first predetermined threshold.

In some embodiments, the procedure performed may be a diagnostic procedure. Alternatively, the procedure performed may be a therapeutic procedure.

Such embodiments of the present invention initiate single or multiple measurements or therapeutic applications when a device comes into contact with tissue without a required action from a user. Such initiation may be conducted automatically. This may significantly reduce the physiological changes that occur due to time and pressure. Data quality may be enhanced and variability due to external factors may be reduced. Detecting the device's proximity to tissue enables the desired diagnostic measurement or therapeutic application to be started upon contact between the tissue and the device. Detecting when the device is in contact with the tissue will also automate the interactions required by the user to initiate a measurement or therapeutic application which requires contact with the tissue. This can automate both a single interaction between the device and the tissue and sequences of multiple or repeated interactions.

### BRIEF DESCRIPTION OF THE DRAWINGS

These and other aspects and features of the present invention will become apparent to those of ordinary skill in the art upon review of the following description of specific embodiments of the invention in conjunction with the accompanying Figures, wherein:

FIG. 1 illustrates an apparatus in accordance with the present invention.

FIG. 2 illustrates a method for initiating a procedure in accordance with the present invention.

FIG. 3 illustrates a method for initiating and conducting a procedure in accordance with the present invention.

FIG. 4 illustrates a data chart in accordance with an embodiment of the present invention.

FIG. 5 illustrates a data chart in accordance with an embodiment of the present invention.

FIG. 6 illustrates a data chart in accordance with an embodiment of the present invention.

FIG. 7 illustrates a data chart in accordance with an embodiment of the present invention.

FIG. 8 illustrates a data chart in accordance with an embodiment of the present invention.

FIG. 9 illustrates a data chart comparing the pressure normalized effect of gentle and firm pressure on hemoglobin concentration, oxygenation percentage, and the PLS %.

FIG. 10 illustrates a data chart comparing the time normalized effect of gentle and firm pressure on hemoglobin concentration, oxygenation percentage, and the PLS %.

FIG. 11 illustrates light reflectivity ratio data in an automated contact detection sequence.

### DETAILED DESCRIPTION OF EMBODIMENTS

The following exemplary embodiments are described below with reference to the figures in the context of detecting tissue contact via a probe to initiate a tissue measurement or other procedure. All exemplary embodiments of the present invention are intended to be used in any applicable field of endeavor. The disclosure of “Method of Detecting Tissue Contact For Fiber-Optic Probes to Automate Data Acquisition Without Hardware Modification,” published Jul. 23, 2013 in Biomedical Optics Express, Vol. 4, No. 8, is hereby incorporated in its entirety.

One exemplary implementation relates to a probe apparatus that is used for optically examining a target for tumors or lesions using what is referred to as “Early Increase in microvascular Blood Supply” (EIBS) that exists in tissues that are close to, but not themselves, the lesion nor tumor. Other exemplary implementations relate to probe apparatus that are used to screen for possibly abnormal tissue using Low-coherence Enhanced Backscattering (LEBS) spectroscopy. While the abnormal tissue can be a lesion or tumor, the abnormal tissue can also be tissue that precedes formation of a lesion or tumor, such as precancerous adenoma, aberrant crypt foci, tissues that precede the development of dysplastic lesions that themselves do not yet exhibit dysplastic phenotype, and tissues in the vicinity of these lesions or pre-dysplatic tissues. In one exemplary implementation, an application is for detection of such lesions in colonic mucosa in early colorectal cancer, but other applications are possible as well.

The methods described herein can be used with the optical probes described, for example, in U.S. patent application Ser. No. 11/604,659 (published as U.S. Patent Application Publication No. 2007/0129615), U.S. patent application Ser. No. 12/684,837 (published as U.S. Patent Application Publication No. 2010/0262020) and U.S. patent application Ser. No. 13/963,560 (published as U.S. Patent Application Publication No. 2014/0036271), the disclosures of which are incorporated herein by reference in their entireties. The remainder of the present description focuses mainly on the methods and apparatus relating to detection of contact of the probe with tissue or close proximity of the probe with tissue.

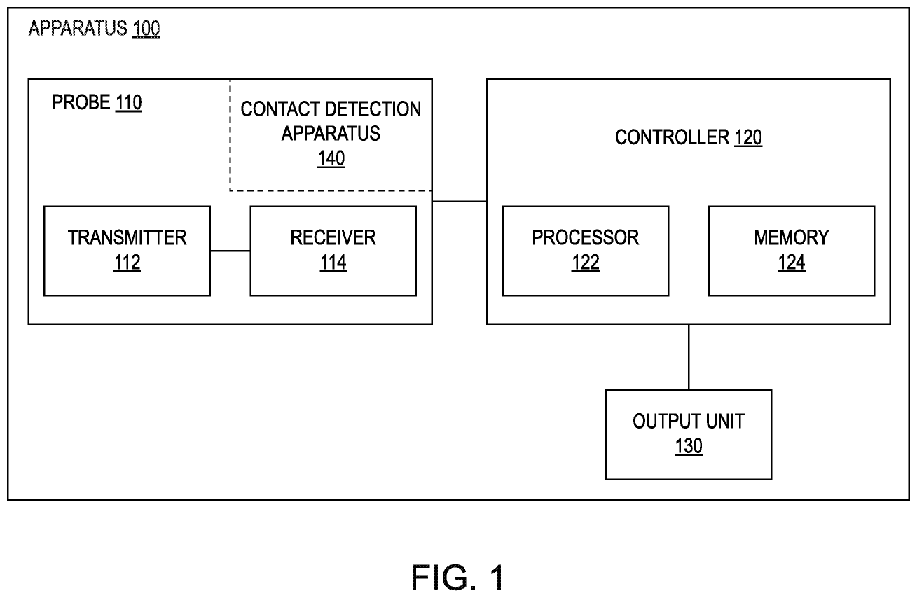

FIG. 1 illustrates an exemplary apparatus for initiating a procedure on tissue in accordance with the present invention. As illustrated in FIG. 1, apparatus 100 includes probe 110, controller 120 and output unit 130. Probe 110 includes transmitter 112 and receiver 114. Controller 120 may include processor 122 and memory 124. Memory 124 may be a non-volatile memory medium.

In exemplary operation of apparatus 100, transmitter 112 transmits a signal that is reflected off of tissue. The reflected/diffused signal is received by receiver 114. The reflected/diffused signal received by receiver 114 may vary from the signal transmitted by transmitter 112 in accordance with physical properties of the tissue and with changes in a proximity of probe 110 to the tissue. Data representing the reflected/diffused signal received by receiver 114 is transferred to controller 120.

Apparatus 100 measures changes in physical properties so as to determine whether probe 110 is in contact with tissue or whether probe 110 is within a predetermined distance of the tissue. In an exemplary embodiment of the present invention, apparatus 100 may measure changes in light intensity. Alternatively, apparatus 100 may measure changes in other physical properties such as resistance, capacitance, inductance, or pressure.

Apparatus 100 may determine whether probe 110 has come in contact with the tissue or whether probe 110 is within a predetermined distance from the tissue. Apparatus 100 may analyze changes in a signal received by receiver 114 of probe 110 that is reflected from the tissue as probe 110 approaches the tissue by comparing the signal to a predetermined threshold. Use of apparatus 100 may decrease a time of contact between probe 110 and the tissue prior to initiation of a measurement or other tissue procedure. Use of apparatus 100 may also reduce the time-dependent impact of pressure applied to the tissue. This will allow a measurement to be taken from the tissue with minimal changes in the physiological parameters that are to be observed.

Controller 120 analyzes the reflected/diffused signal to determine whether probe 110 is within a predetermined distance from the tissue. The predetermined distance may be stored as data within memory 124. Predetermined data, past signal data, or other information or thresholds may be stored within memory 124 for use in analysis performed by controller 120. Processor 122 may be utilized in any analysis performed by controller 120 upon the reflected/diffused signal.

When controller 120 determines that probe 110 is within the predetermined distance to the tissue, controller 120 may initiate a measurement or other procedure upon the tissue. The procedure may be a diagnostic procedure, a therapeutic procedure, or other type of procedure or medical procedure. The measurement or procedure performed on the tissue may be performed by apparatus 100. Probe 110 may be utilized in the measurement or the procedure. Transmitter 112 and/or receiver 114 may be utilized in the performance of the measurement or the procedure. Signals transmitted by transmitter 112 and signals received by receiver 114 may be used by controller 120 for determination of proximity of probe 110 to the tissue for determination of initiation of the procedure, and/or for the procedure itself. Alternatively, the procedure may be performed by a separate device, i.e. apparatus 100 is not utilized in the performance of the procedure.

When controller 120 determines that probe 110 is within the predetermined distance to the tissue, controller 120 may output an instruction via output unit 130 to an operator of apparatus 100 to move probe 110 away from the tissue. The instruction output by output 130 may be an audio, visual or any other form of data output or communication. Output unit 130 may include a speaker, display screen, display medium or other audio and/or visual output mechanism.

When controller 120 determines that probe 110 is within the predetermined distance to the tissue, controller 120 may continue to analyze the signals received by receiver 114 as probe 110 is being moved toward the tissue or being moved away from the tissue to determine when probe 110 is out of contact with the tissue. Alternatively, controller 120 may then analyze the signal received by receiver 114 to determine when probe 110 is beyond the predetermined distance to the tissue. Controller 120 may output via output unit 130 an indication that probe 110 is out contact with the tissue, and/or beyond the predetermined distance to the tissue.

In an exemplary embodiment, apparatus 100 may continuously and rapidly monitor the intensity of signals reflect/diffuse from the tissue. This is accomplished by continuously and rapidly transmitting a signal by transmitter 112 and continuously and rapidly receiving a signal that is reflected from the tissue by receiver 114. Data corresponding to signals received by receiver 114 may be stored within memory 124. The intensity of the signals received may also be stored within memory 124. The intensity of the received data points may be stored individually or may be averaged over data points to minimize the impact of noise or invalid readings. The average intensity may then be compared by controller 120 to a predetermined threshold stored within memory 124. Controller 120 then determines if probe 110 is within a predetermined distance to the tissue. Alternatively, controller 120 may determine if probe 110 is in contact with the tissue. If the average intensity is below the threshold, new measurements are taken. If the average intensity is above the threshold, controller 120 determines that probe 110 is within a predetermined distance to the tissue and/or in contact with the tissue, and controller 120 then starts an operation to begin a procedure. Alternatively, controller 120 may determines if probe 110 is in contact with the tissue.

In another embodiment, apparatus 100 may be utilized in conjunction with an additional signal detection circuitry. Apparatus 100 may be utilized in a continuous detection mode to confirm positioning of probe 100 relative to the tissue. Such continuous detection may determine probe contact with the tissue as well as confirm data stability during acquisition of tissue data used for processing and evaluation in a procedure.

In an alternative embodiment of the present invention, apparatus 100 may include contact detection apparatus 140 (shown by broken lines in FIG. 1). Contact detection apparatus 140 may comprise a physical sensor integrated into probe 110. Contact detection apparatus 140 may transmit a signal when a probe tip of contact detection apparatus 140 comes in contact with the tissue. In such an embodiment, controller 120 would compare the signal of contact detection apparatus 140 to a predefined threshold, and if contact detection apparatus 140 indicated contact, controller 120 would use a hardware trigger to start acquisition of signal data from receiver 114 in parallel to the data of contact detection apparatus 140. It is estimated that such a modified apparatus 100 could result in a start of data acquisition in as little as 20 ms to 30 ms from probe-to-tissue contact.

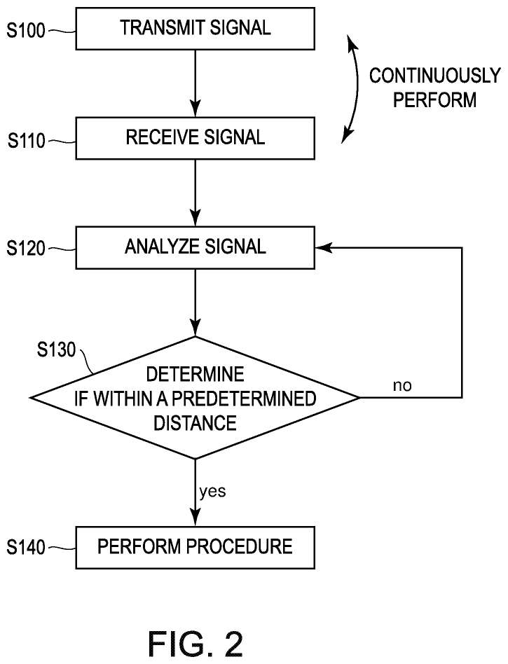

FIG. 2 illustrates a method for initiating a procedure in accordance with apparatus 100. In step S100, transmitter 112 transmits a signal, preferably directed toward the tissue. In step S110, receiver 114 receives a signal reflected from the tissue. Steps S100 and S110 may be rapidly and continuously performed throughout the performance of the method. In step S120, controller 120 analyzes the reflected/diffused signal received by receiver 114. Controller 120 performs the analysis by comparing the reflected/diffused signal to the transmitted signal. Alternatively, controller 120 may analyze the reflected/diffused signal by comparing the reflected/diffused signal to a predetermined threshold.

In step S130, controller 120 determines, based upon the analysis performed in step S120, whether probe 110 is in contact with the tissue or whether probe 110 is within a predetermined distance of the tissue. If controller 120 determines that probe 110 is in contact with the tissue or otherwise within a predetermined distance of the tissue, controller 120 proceeds to step S140. In step S140, controller 140 begins performance of the tissue measurement or procedure. If controller 120 determines that probe 110 is not within a predetermined distance of the tissue, controller 120 returns to step S120. Alternatively, in step S140, controller 120 may determine whether probe 110 is in contact with the tissue.

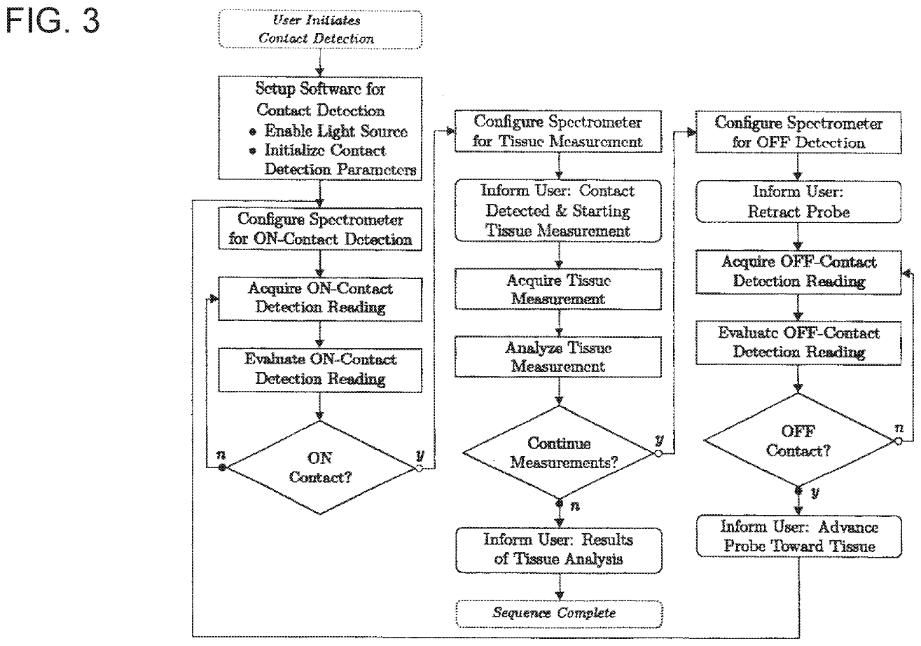

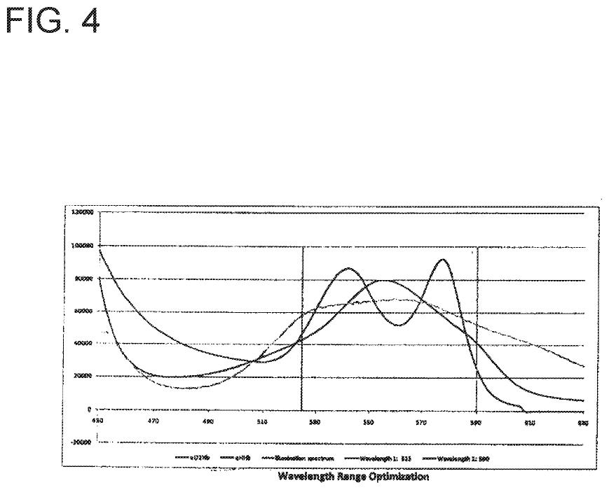

FIG. 3 illustrates a method for initiating and conducting a procedure in accordance with the present invention.

Apparatus 100 may detect contact between the tissue and probe 110 by continuously and rapidly monitoring the reflected/diffused light intensity from the tissue and then evaluating the data against predetermined criteria. To begin, the method is initiated by setting the contact detection parameters. In an exemplary embodiment, a complete contact sequence to trigger data acquisition, i.e. a procedure, may require 5 consecutive readings above a predetermined threshold and variability. However, other embodiments may require a different number of consecutive readings or require a running average to reach a predefined value. Once this process is completed, apparatus 100 may switch modes to perform a tissue measurement or procedure. In alternative embodiments, additional components may be utilized to perform the tissue measurement or procedure.

After controller 120 determines that the procedure is competed, controller 120 may output via output unit 130 an indication of completion of the procedure. The indication of completion of the procedure may be an output to a user of apparatus 100 to indicate that probe 110 may be moved away from the tissue. Alternatively, the indication of completion may be a data signal that initiates automatic movement of probe 110 away from the tissue. Controller 120 may again analyze the signal received by receiver 114 by comparing the reflected/diffused signal to the transmitted signal. Alternatively, controller 120 may analyze the reflected/diffused signal by comparing the reflected/diffused signal to a predetermined threshold.

When controller 120 determines that probe 110 is beyond a predetermined distance from the tissue, controller 120 may output via output unit 130 an indication that probe 110 is beyond a predetermined distance from the tissue and/or out of contract with the tissue.

Parameters for determining contact between the probe and the tissue may include: (1) specific wavelength range (if the probe is an optical probe); (2) normalized threshold intensity ratio; (3) consistent threshold value (for example, within 3% for 5 consecutive measurements); and (4) integration time.

Additional analysis for determining contact between the probe and the tissue may include evaluating an angle between the probe tip and the tissue. As a function of distance, angles as large as 45-60 degrees from normal to the tissue surface may not affect the reflected intensity. However, more extreme angles (e.g., probe tangential to tissue surface) may cause fluctuations in reflected intensity that resemble the probe sliding along the tissue surface.

In one embodiment of the present invention, probe 110 may comprise an optical probe, transmitter 112 may comprise an optical transmitter, and receiver 114 may comprise an optical receiver. For example, receiver 114 may comprise one or more spectrometer(s). The optical probe may include an illumination source. In such an embodiment, apparatus 100 is used to record the intensity of light reflected from a tissue sample. Spectrometers within probe 110 may record the light reflected from the tissue. Transmitter 112 may include one or more illumination channels and receiver 114 may include one or more collection channels (typically 2 or 3 collection channels). The distal tip of probe 110 may include a plurality of thin film polarizers to polarize the incident light and to enable collections of co-polarized, I∥(λ), and crosspolarized, I_(λ), signals. Probe 110 may record optical data reflected from the tissue spanning the wavelength range of 350 to 700 nm, however, probe 110 is not limited to such a range. For example, the probes described in the above-incorporated U.S. Patent Application Publication No. 2007/0129615 and U.S. Patent Application Publication No. 2010/0262020 may be used.

In such embodiments, apparatus 100 may monitor the reflected/diffused light intensity for tissue contact and record light that is received in a wavelength around 525 nm, however, apparatus 100 is not limited to such a wavelength. For example, transmitter 112 may transmit light with a wavelength outside of the visible spectrum. Using light that is not visible may serve to minimize any impact of ambient light that may be present due to any external device or other influence (such as video devices or an endoscope).

Apparatus 100 may collect, for example, 10 pixels of data from probe 110. The intensity of the received light may be averaged over the 10 pixels of data to minimize the impact of electrical noise. Controller 120 may detect contact of probe 110 with the tissue by reading repeated measurements of the reflected/diffused light and comparing the data to a predetermined threshold that is acquired. When acquiring data to detect tissue contact, it may be desirable to take optical readings as quickly as possible.

In order to minimize the time it takes to acquire the data, only one light receive channel of data from the probe may be acquired. To further facilitate a rapid light measurement, only a small wavelength range of reflected/diffused light may be acquired. One embodiment may use reflected/diffused light around 525 nm. This wavelength range of reflected/diffused light that is acquired has been chosen in order to maximize the light output from the illumination source while minimizing the light absorption from hemoglobin that may be present in the tissue as well as minimizing the impact of any ambient light present during the measurement, such as from the endoscope light. Alternatively, receiver 114 may include a single spectrometer that is configured to record reflected/diffused light intensity at different wavelengths within memory 124 in order to minimize the processing time required to record a larger spectrum while providing the ability to capture changes that occur at different wavelengths in the spectrum.

In another embodiment of the present invention, a sum of the intensities of multiple channels or wavelength ranges above a given baseline may be used to determine when probe 110 is in contact with the tissue or otherwise within a predetermined distance of the tissue. In order to amplify changes in the spectrum of the signal received by receiver 114, controller 120 may use a product of the intensities of signals received in multiple channels of receiver 114 that above a given baseline in order to determine when probe 110 is in contact with the tissue.

When starting the process to detect contact with the tissue, controller 120 may configure receiver 114 for the specific wavelength range (525 nm) and integration time (20 ms) that will be used. The integration time that is used depends on a number of factors, such as the optical fiber parameters, illumination source intensity, tissue type, and the sensitivity of the spectrometers. The integration time is selected to be as short as possible while still providing an adequate optical signal to be able to differentiate the light reflected off of the tissue from the illumination source from noise caused by the detection circuitry or any background light that may be present.

Once apparatus 100 is configured for the tissue contact detection data acquisition, controller 120 may initiate the tissue contact detection method, as previously discussed. Controller 120 may include a state machine that provides a sequencing of steps necessary to acquire the reflected/diffused light readings for both the tissue contact detection measurements as well as the tissue physiological measurements. The state machine may include states such as setup, configuring spectrometers for contact detection, acquiring contact detection, evaluating contact detection, configuring spectrometers for tissue measurement, acquiring tissue measurement, evaluating tissue measurement, configuring spectrometers for probe retraction, acquiring probe retraction, evaluating probe retraction, and a complete state. Such processes may involve initiating an optical recording by the spectrometer(s). This data acquisition can be started using a hardware trigger. The hardware trigger is particularly helpful when more than one spectrometer is used in data acquisition in order to synchronize the start time of data acquisition between the two light receive channels (and spectrometers).

Once apparatus 100 completes the tissue contact detection data acquisition and the recorded optical data is read, the optical data may be evaluated to determine if the average intensity of the reflected/diffused light from the tissue around 525 nm is greater than a predefined threshold. If the average intensity is greater than the predefined threshold, the probe is determined to be in contact with the tissue or within a predetermined distance of the tissue.

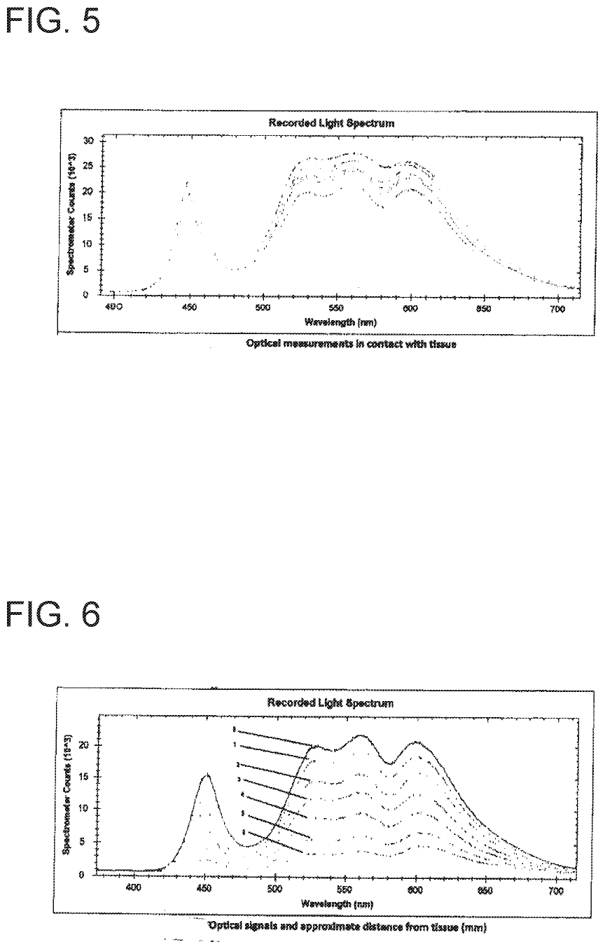

FIGS. 5 and 6 are data charts illustrating an increase in the reflected/diffused light from the tissue received by the probe as it approaches the tissue. As illustrated in FIGS. 5 and 6, a threshold of 12,000 counts may be used to detect when probe 110 is approaching contact with the tissue. This threshold allows detection when probe 110 is within approximately 2 mm of the tissue. The time for probe 110 to travel the remaining distance to result in good contact with the tissue is typically around 100 ms. This is less than the time that apparatus 100 will take to setup and acquire the tissue measurement (approximately 150 ms).

The threshold value may be adjusted to fine tune the contact detection algorithm (the distance at which contact is detected and the tissue measurement is triggered). The threshold values may also be adjusted to account for a sensitivity of the spectrometers used as well as the transmission properties of probe 110. In other embodiments, alternative comparisons of the acquired reflected/diffused light may be performed.

In one embodiment, if an average intensity of the reflected/diffused light from the tissue is around 525 nm and is less than the predefined threshold, then controller 120 determines that probe 110 is not in contact with the tissue. Controller 120 may then initiate another optical reading from receiver 114. The time between the start of one tissue contact detection optical reading and the beginning of another optical reading in this embodiment would be no more than approximately 170 ms. Significantly shorter times are possible with different spectrometers or hardware configurations.

If controller 120 determines that probe 110 is in contact with the tissue, controller 120 will perform any events that are waiting for the tissue contact to occur. This may include processes that begin steps necessary to start a procedure or other tissue measurement. This may also include configuring receiver 114 to receive alternative signals or light in a different wavelength range.

When the reflected/diffused light from the tissue has been acquired by receiver 114, controller 120 may perform analysis or other activities on the data. Such activities may include: storing the recorded data signals reflected from the tissue in memory 124, outputting the received data signals via output unit 130 on a screen for user interpretation, evaluating the light reflected from the tissue to determine if the light data is usable by a physician and/or a data processing algorithm, processing the data to extract physiological data from the received data signals and displaying the physiological data (via output unit 130) to a user for interpretation. When data acquisition and analysis by controller 120 is compete, controller 120 via output unit 130 may inform a user by an audible tone and/or a visual indication on a monitor to retract probe 110 from the tissue.

In one embodiment, data acquisition may start between 170 ms and 330 ms from probe 110 to tissue contact. Such a potential delay (up to 330 ms) is significantly less than without the use of apparatus 100, i.e., a human initiating the process. Such a delay may be further reduced via use of precise hardware components (such as a different spectrometers, different spectrometer communication interface, or a different control computer) if necessary. This also provides a more consistent tissue contact to data acquisition starting time span (at most 160 ms of variability). Further increases in processing speeds may also decrease these data acquisition delays.

Controller 120 may automatically detect if probe 110 is out of contact with the tissue once the tissue measurement or procedure is complete. This may be done by repeating the method used for tissue contact detection and looking for the reflected/diffused light signal to fall below a given threshold (or decrease by a predetermined percentage). In such an embodiment, controller 120 may operate for the reflected/diffused light intensity at a wavelength around 525 nm with a predetermined threshold of 7,000 counts; however, such a threshold value may be adjusted. Such a threshold may be set lower than the tissue contact detection threshold in order to prevent vacillation between detection of probe 110 being in contact and out of contact as well as ensuring that probe 110 is sufficiently out of contact in order to prevent premature measurements due to poor tissue contact.

When controller 120 has determined that probe 110 is sufficiently out of contact from the tissue, output unit 130 may output to a user via an audible and/or visual indicator, instructing to initiate contact with the tissue to acquire another measurement. At this point in time, controller 120 may automatically repeat the tissue contact detection process.

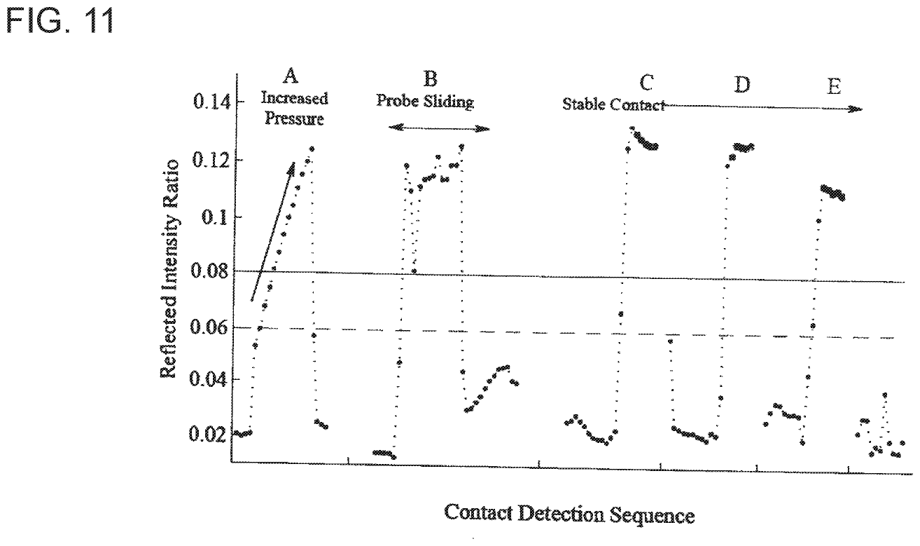

FIG. 11 illustrates light reflectivity ratio data in an automated contact detection sequence. As illustrated in FIG. 11, threshold ratios (tissue/calibration) where ON-contact ≥0:08 and OFF-contact <0:06. This ON-contact threshold detects when the probe is within 2 mm of the tissue. In other words, the solid line at a ratio of 0.08 indicates ON-contact and the dashed line at a ratio of 0.06 indicates OFF-contact. The time required for the probe to travel the remaining distance and establish stable contact with the tissue is typically <100 ms. This is less than the time needed by the system software to setup for tissue measurement acquisition (around 150 ms). The threshold values may be easily be adapted to account for the sensitivity of different spectrometers, as well as the transmission properties of different probes.

In FIG. 11, each of the data points represent the reflected intensity ratio recorded as the probe was placed in contact with tissue in 3 isolated sequences, labeled A-E. Points connected by the dotted line are continuous with a sampling rate of 170 ms, and gaps represent larger time lapses. The solid line is the ON-contact threshold (0.08) and the dashed, line is the OFF-contact threshold (0.06). Sequence A represents the probe slowly advancing toward the mucosa, and once in contact with tissue, pressure was continually applied to the probe. Sequence B indicates the probe sliding along the tissue surface. The probe was not held steady and the reflected intensity values are highly variable. In these two sequences, the reflected intensity rises above the threshold, but there is no consistency in the readings. The last sequence (C-E) models the ideal performance where the reflected intensity rises sharply above the threshold (indicating contact with tissue) and maintains a constant value (indicating steadiness of the probe). The circled points designate data that meets all criteria for good, stable contact and triggered a tissue measurement. The gap in time sampling immediately following the circles points (approximately 600 ms) is due to exiting contact detection mode, entering acquisition mode and reentering contact detection mode. After data acquisition, the probe is retracted and the reflected intensity drops dramatically below the OFF-contact threshold.

Alternative embodiments of apparatus 100 that utilize an optical transmitter may be used. Such embodiments include optical transmitters and receivers that use different wavelength ranges of light. Such ranges may be broader or narrower than the wavelength range previously described. Additional embodiments may be used alone or in combination with other embodiments of the present invention.

In another embodiment of the present invention, an optical signal may be evaluated by controller 120 to determine if one or more optical channels of receiver 114 have a received intensity above a threshold for a particular wavelength or wavelength range. Such an embodiment may utilize a wavelength where the illumination source outputs high signal intensity relative to ambient light. When the received light is above a given threshold, probe 110 may be determined to be in contact with the tissue, i.e. the increase in the light from transmitter 112 is reflected back by the tissue. If more than one optical channel of receiver 114 is used to evaluate the received light intensity, separate trigger thresholds may be used depending on the characterizations of the expected light from the respective channels. These thresholds may be general thresholds for each light receive channel or they may be probe specific. The probe specific thresholds may be configured based on manufacturing test results or based on a calibration performed before each data acquisition use.

In yet another embodiment of the present invention, an optical signal received by receiver 114 may be evaluated by using a ratio of the optical signal received between multiple channels for a given wavelength or wavelength range. Such data is illustrated in FIG. 8. Such an embodiment may be useful with polarization gated probes when the interaction of light with tissue alters the polarization of the reflected light. A difference in the recorded data from the receive channels of receiver 114 is due to the difference in polarization of the reflected/diffused light collected from the tissue by the two channels. As illustrated in FIG. 8, channel 1 is used to collect the co-polarized signal while channel 2 is used to collect the cross-polarized signal. The polarization difference provides the ability for depth-selective tissue analysis. When the ratio of the co-polarized received light to the cross-polarized received light is above a given threshold, probe 110 may be determined to be in contact with the tissue. FIG. 8 illustrates how a ratio of the signals received by two channels may be used to predict contact between the probe and the tissue.

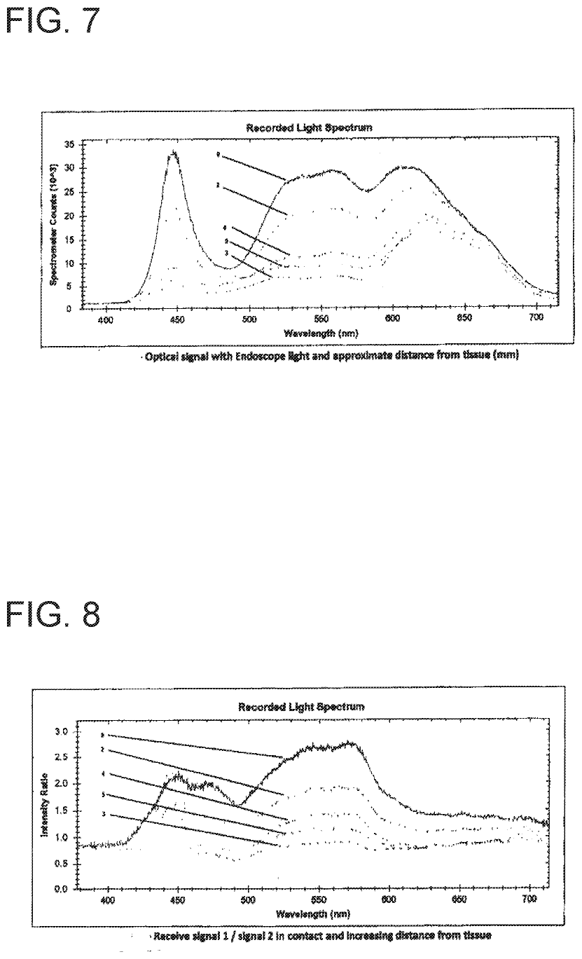

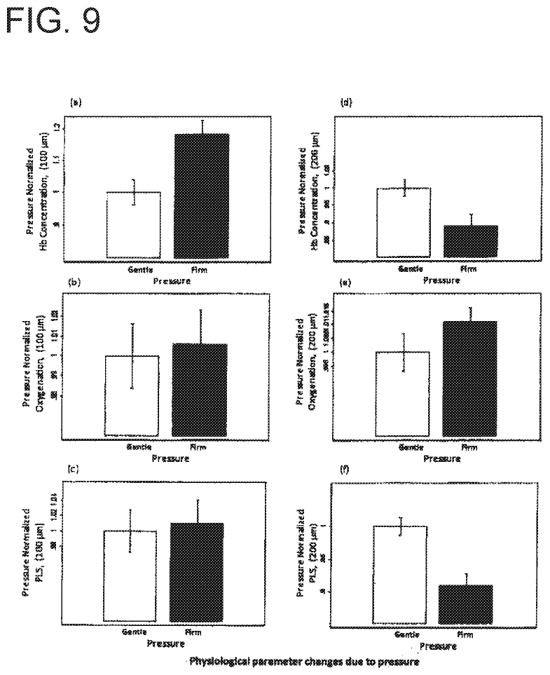

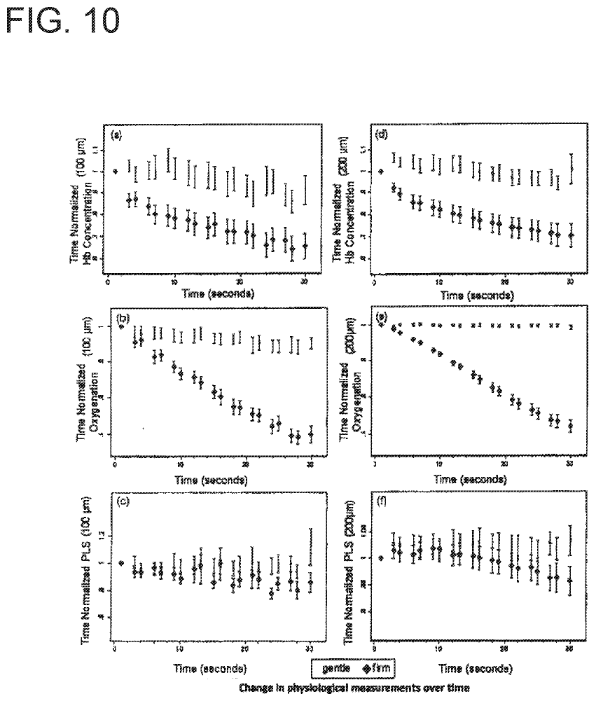

In another embodiment of the present invention, controller 120 may use light intensity recorded at a wavelength when transmitter 112 is of low illumination intensity and there is a high ambient light intensity, such as approximately 650 nm as illustrated in FIG. 7. When the ambient light that is recorded decreases below a given threshold, probe 110 may be determined to be in contact with the tissue and thus blocking the ambient light from being reflected into the optical receive channels. Because this embodiment relies on ambient light, this embodiment may be used with only a single receive channel and no light transmission channel.

In another embodiment of the present invention, controller 120 may evaluate the intensity of the reflected/diffused light from one or more receive channels with a preset baseline, for example, a spectrometer electrical baseline reading. A predefined percentage increase in the intensity of the light received over the baseline may be used to determine when probe 110 is in contact with the tissue which will result in the beginning of a procedure or other type of tissue measurement.

In another embodiment of the present invention, receiver 114 may include multiple optical receive channels to record the received light intensity at different wavelengths in order to minimize the processing time required to record a larger spectrum while providing the ability to capture changes that occur at different wavelengths in the spectrum. For example, a first channel may be used to acquire data with a high system illumination signal and a low expected ambient light component while a second channel may be used to acquire data with a low system illumination source signal and a high expected ambient light component. When both the reflected/diffused light from the system illumination source increases above a threshold and the ambient light signal decreases below a threshold, controller 120 determines that tissue contact has occurred. In such an embodiment, the multiple light intensity values may be recorded or stored within memory 124.

In another embodiment of the present invention, multiple channels of a polarization gated probe may be utilized to evaluate a difference between the reflected/diffused light received by the channels of receiver 114 in order to determine when probe 110 is in contact with the tissue. The characteristics of the co-polarized and cross-polarized channels of probe 110 provide an ability for depth-selective tissue analysis and are such that the relative difference between the spectra may be used to determine when probe 110 is in contact with the tissue by evaluating the interaction of the reflected/diffused light with the superficial layer of the tissue in contact.

In an alternative embodiment of the present invention, a pressure sensor may be integrated into a distal end of probe 110. Controller 120 may monitor the pressure sensor to determine if probe 110 is in contact with the tissue. When the pressure is measured to be greater than a predefined threshold, controller 120 may initiate a measurement of the light received by receiver 114. Controller 120 may also record the pressure applied to the tissue during the measurement in memory 124.

In other embodiments of the present invention, probe 110 may measure resistance, inductance, capacitance, or pulsing air. A sensor that measures the respective physical property may be integrated into the distal end of probe 110 to provide information to controller 120 so that controller 120 may determine if probe 110 is in contact with the tissue.

The foregoing description of the exemplary embodiments of the invention has been presented only for the purposes of illustration and description and is not intended to be exhaustive or to limit the invention to the precise forms disclosed. Many modifications and variations are possible in light of the above teachings.

What is claimed is:

1. A method of initiating a procedure performed by a probe on tissue, the method comprising:
(a) obtaining a signal from the probe, the signal varying with changes in a proximity of the probe to the tissue;
(b) analyzing the obtained signal by a controller to determine whether the probe is within a predetermined distance of the tissue; and
(c) the controller initiating the procedure when it is determined that the probe is within the predetermined distance of the tissue.

2. The method according to claim 1, wherein steps (a) and (b) are repeated until the probe is determined to be within the predetermined distance of the tissue.

3. The method according to claim 1, wherein step (b) includes comparing the obtained signal to a predetermined threshold.

4. The method according to claim 3, wherein the probe is determined to be in close proximity of the tissue when a value of the obtained signal is greater than the predetermined threshold.

5. The method according to claim 1, wherein the probe includes a receiver that measures a physical property to output the obtained signal.

6. The method according to claim 5, wherein the receiver also is used to perform the procedure.

7. The method according to claim 6, wherein the procedure is a diagnostic procedure.

8. The method according to claim 5, wherein the receiver is an optical receiver and the physical property is a light intensity.

9. The method according to claim 5, wherein the receiver is an electrical receiver and the physical property is a resistivity.

10. The method according to claim 5, wherein the receiver is a mechanical receiver and the physical property is a pressure.

11. The method according to claim 3, wherein
the probe transmits light onto the tissue and receives reflected/diffused light from the tissue to obtain the signal,
the signal represents an intensity of the reflected light, and
in step (b), the controller compares the obtained signal to a predetermined threshold intensity value.

12. The method according to claim 3, wherein
the probe transmits a first polarized light and a second polarized light different from the first polarized light onto the tissue and receives a first reflected/diffused light and a second reflected/diffused light different from the first reflected/diffused light from the tissue,
the obtained signal represents a ratio between the first reflected/diffused light and the second reflected light, and
in step (b), the controller compares the obtained signal to a predefined threshold ratio value.

13. The method according to claim 11, wherein
the probe receives the reflected/diffused light at multiple wavelengths, and
the signal represents an average of intensity values that are obtained from the probe according to the reflected/diffused light at the multiple wavelengths.

14. The method according to claim 1, further comprising:
(d) after the controller determines that the procedure has been completed, the controller indicates that the procedure has been completed to cause an operator of the probe to move the probe away from the tissue;
(e) the controller analyzes the signal as the probe is being moved away from the tissue to determine when the probe has come out of contact with the tissue; and
(f) the controller indicates to the operator that the probe has come out of contact with the tissue.

15. The method according to claim 14, wherein
in step (b), the controller compares the obtained signal to a first predetermined threshold, and
in step (d), the controller compares the obtained signal to a second predetermined threshold that is different from the first predetermined threshold.

16. The method according to claim 1, wherein the procedure performed in step (c) is a diagnostic procedure.

17. The method according to claim 1, wherein the procedure performed in step (c) is a therapeutic procedure.

18. An apparatus for initiating a procedure on tissue, the apparatus comprising:
a probe including a transmitter that transmits a signal and a receiver that receives a reflected/diffused signal, the reflected/diffused signal varying from the transmitted signal with changes in a proximity of the probe to the tissue; and
a controller that analyzes the reflected/diffused signal to determine whether the probe is within a predetermined distance from the tissue, wherein
the controller initiates the procedure when it is determined that the probe is within the predetermined distance of the tissue.

19. The apparatus according to claim 18, wherein the controller includes a processor and a memory.

20. The apparatus according to claim 18, further comprising an output unit, wherein
when the controller determines that the procedure has been completed, the controller:
(i) outputs via the output unit an instruction to an operator of the apparatus to move the probe away from the tissue,
(ii) analyzes the signal as the probe is being moved away from the tissue to determine when the probe has come out of contact with the tissue, and
(iii) outputs via the output unit an indication to the operator that the probe has come out of contact with the tissue.
